(19) United States
(12) Patent Application Publication
(10) Pub. No.: US 2016/0362355 A1
(43) Pub. Date: Dec. 15, 2016

(54) Method of Fabricating Oil Product of Gasoline

(71) Applicant: Institute of Nuclear Energy Research, Atomic Energy Council, Executive Yuan, R.O.C., Taoyuan County (TW)

(72) Inventors: Kuo-Chao Liang, Taoyuan County (TW); How-Ming Lee, Taoyuan County (TW); Shiaw-Huei Chen, Taoyuan County (TW); Feng-Mei Yeh, Taoyuan County (TW); To-Mei Wang, Taoyuan County (TW); Meng-Han Huang, Taoyuan County (TW); Lieh-Chih Chang, Taoyuan County (TW); Chin-Ching Tzeng, Taoyuan County (TW)

(21) Appl. No.: 14/737,584

(22) Filed: Jun. 12, 2015

(51) Int. Cl. C07C 41/01; C07C 41/34; C10J 3/82; C10K 1/00; C10L 1/06; C10G 3/00

(52) U.S. Cl. CPC C07C 41/01; C10L 1/06; C10G 3/42; C10J 3/82; C10K 1/003; C10K 1/004; C07C 41/34; C10L 2200/0492; C10L 2290/04; C10L 2290/08; C10L 2290/42; C10L 2290/543; C10J 2300/0916; C10J 2300/0969; C10J 2300/0973; C10J 2300/0959; C10J 2300/0956; C10J 2300/1656

(57) ABSTRACT

An oil product of gasoline is fabricated. The product contains hydrocarbon compound ranged as a gasoline composition. The purification process of dimethyl ether (DME) used in the present invention greatly reduces the feed rate for obtaining a smaller reactor with cost down. Carbon dioxide (CO₂) is separated to be recycled back to the gasifier to be reused, archived or used otherwise for improves global environment. At the same time, CO₂ is reacted with hydrocarbons, water vapor, etc. through a novel high-temperature plasma torch to generate a synthesis gas (syngas) of carbon monoxide (CO) and hydrogen (H₂) for regulating a hydrogen/carbon ratio of a biomass- or hydrocarbon-synthesized compound and helping subsequent chemical synthesis reactions. In the end, the final gasoline production has a high yield, a high octane rate, low nitrogen and sulfur pollution and a highly ‘green’ quality.

### TECHNICAL FIELD OF THE INVENTION

The present invention relates to fabricating an oil product; more particularly, relates to fabricating a gasoline-range oil product of hydrocarbons.

### DESCRIPTION OF THE RELATED ARTS

As global climate change and peak oil are happening, alternative energy sources and renewable energy research and development are flourishing. Biomass energy has a reservation as high as the fourth among the world's primary energies, where only oil, coal and natural gas have higher reservations. Concerning biomass fuel, the global annual production value is more than 85 billion U.S. dollars. Hence, liquid fuels converted from biomass have become hottest R&D focus in recent years.

Traditional methods for synthesizing liquid hydrocarbon fuels include: (1) using Fischer-Tropsch (FT) catalyst for directly converting a synthesis gas (syngas) into hydrocarbons; and (2) converting a syngas into methanol at first, dehydrating some of the methanol into dimethyl ether (DME), and then dehydrating the methanol and the DME again to produce synthetic gasoline (i.e. a procedure of methanol to gasoline, MtG, revealed by Exxon Mobil, Co.)

The traditional FT synthesized hydrocarbons has an extremely wide carbon number distribution and the carbon chains are mostly straight, which need downstream refining procedures to increase the proportion of side chains and aromatic products for producing high-quality synthetic gasoline. The Exxon Mobil's MtG procedure has a selectivity rate of the gasoline-range product up to about 80%, and can produce high-octane gasoline. However, due to the thermodynamic limitation of the methanol procedure, the one-step syngas conversion rate is low. A large amount of unreacted reactants has to be flown back and power consumption is increased. Besides, methanol has to be dehydrated into DME at first to be directed to a gasoline reactor for producing gasoline, which makes the whole procedure a little complicating.

Therefore, Haldor Topsoe Co. proposed a one-step TIGAS procedure for DME to overcome the above shortcomings of MtG on producing methanol. However, the patent did not separate DME and carbon dioxide (CO₂). Even though the manufacturing procedure was simplified and equipment cost was low, the operating load of the catalytic gasoline reactor would be increased. The purity of the liquefied petroleum gas (LPG) in the end was not high, which reduced the overall value of the manufacturing procedure.

Hence, the prior arts do not fulfill all users' requests on actual use.

### SUMMARY OF THE INVENTION

The main purpose of the present invention is to reduce the feed rate to the reactor for reducing the required volume of the reactor and lowering the hardware investment.

Another purpose of the present invention is to recycle separated CO₂ back to gasifier for reuse, storage or other use so that carbon reduction is achieved and global climate change is hindered.

Another purpose of the present invention is to fabricate a gasoline with high yield and high octane rate as a high-quality green fuel without pollution of nitrogen and sulfur.

To achieve the above purposes, the present invention is a method of fabricating an oil product of gasoline, comprising steps of (a) forming a raw synthesis gas (syngas) with a biomass and an oxidizer through plasma-assisted gasification; (b) removing acidic gases and compounds containing nitrogen, sulfur and/or chlorine from the raw syngas to form another syngas; (c) using the syngas of hydrogen/carbon monoxide (H₂/CO) to obtain dimethyl ether (DME) through a one-step synthesis with a synthesis catalyst; (d) separating carbon dioxide (CO₂) from DME to obtain purified DME while flowing back unreacted part of the syngas of H₂/CO; (e) processing dehydration to the purified DME in a reactor to obtain water (H₂O), gasoline-range hydrocarbon compounds and light hydrocarbon compounds; and (f) separating H₂O, the gasoline-range hydrocarbon compounds and the light hydrocarbon compounds through condensation to obtain liquid-phase materials of H₂O and gasoline and gas-phase materials of light hydrocarbon compounds, where a part of the gas-phase light hydrocarbon compounds is flown back to process step (e) to increase selectivity rate of the gasoline-range hydrocarbon compounds. Accordingly, a novel method of fabricating an oil product of gasoline is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the basic flow view showing the preferred embodiment according to the present invention;

FIG. 2 is the view showing the state-of-use of the preferred embodiment; and

FIG. 3 is the view showing the product distributions as compared to the prior arts.

### DESCRIPTION OF THE PREFERRED EMBODIMENT

The following description of the preferred embodiment is provided to understand the features and the structures of the present invention.

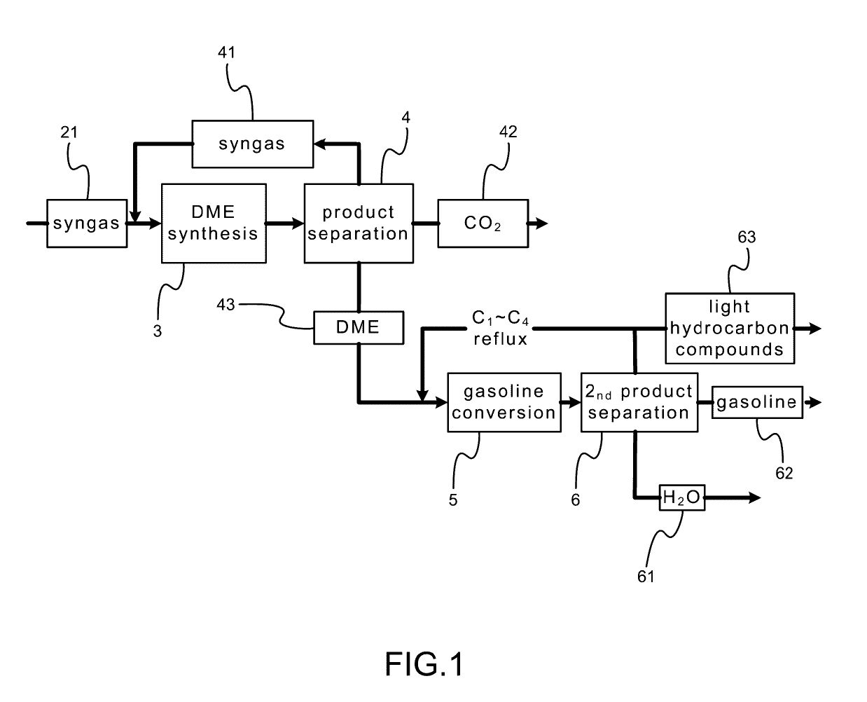

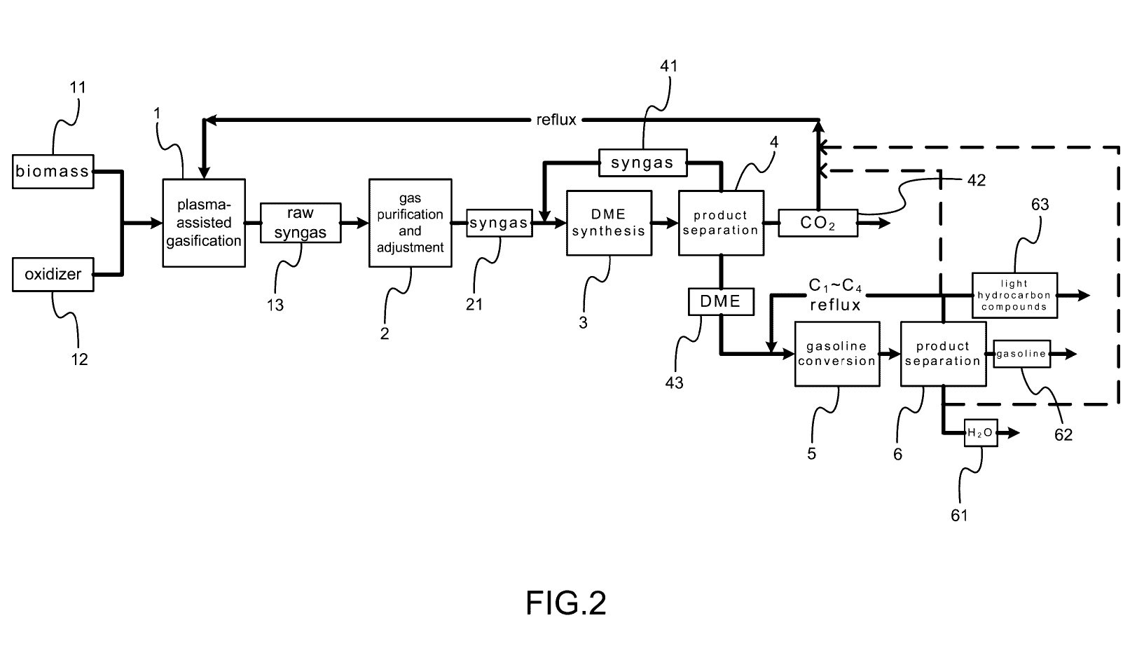

Please refer to FIG. 1 to FIG. 3, which are a basic flow view showing a preferred embodiment according to the present invention; a view showing a state-of-use of the preferred embodiment; and a view showing product distributions as compared to prior arts. As shown in the figures, the present invention is a method of fabricating an oil product of gasoline, comprising steps of (c) synthesizing dimethyl ether (DME) 3; (d) separating out product 4; (e) converting gasoline 5; and (f) further separating out product 6. Before step (c) of synthesizing DME, step (a) and step (b) are further processed, where step (a) is a process of plasma-assisted gasification 1 and step (b) is a process of gas purification and adjustment 2.

(a) Plasma-assisted gasification 1: A biomass material 11 (such as a hydrocarbon compound of biomass or coal) and an oxidizer 12 are used to obtain a raw synthesis gas (syngas) 13 with a plasma-assisted gasifier. Therein, the temperature in the gasifier is controlled through the power level of a plasma torch and the supply amount of the oxidizer 12 and is set between 850 celsius degrees (° C.) and 1400° C.; the oxidizer 12 is carbon dioxide (CO₂), water (H₂O), oxygen (O₂), air or a mixture thereof; and the raw syngas 13 is a mixture of carbon monoxide (CO), hydrogen (H₂), CO₂ and H₂O.

(b) Gas purification and adjustment 2: Acidic gases and compounds like nitrogen, sulfur and chlorine are removed from the raw syngas 13 until a syngas 21 is obtained with CO₂ less than 10 vol. %, nitrogen less than 1 ppm, sulfur less than 1 ppm, chlorine less than 1 ppm and a molar ratio of hydrogen/carbon monoxide (H₂/CO) at 0.7˜2.5.

(c) Synthesizing DME 3: The syngas 21 with the 0.7˜2.5 molar ratio of H₂/CO is directed to a synthesizer for obtaining DME through a one-step synthesis with a catalyst. Therein, a temperature for reaction is set at 200° C.˜300° C. and a pressure for reaction is controlled at 30 atm˜60 atm. Thus, a breakthrough for the thermodynamic limit of methanol synthesis is made while a reactor for methanol dehydration is saved.

(d) Separating out product 4: A first and a second distillation columns (or similar devices) are used to separate CO₂ 42 from DME 41. The first distillation column 41 is used to flow unreacted part of the syngas 21 back to process step (c); and, the second distillation column 42 is used to separate CO₂ 42 to obtain purified DME 43. Therein, the separated CO₂ 42 has a molar ratio of flow not lower than 90% of a molar ratio of flow of CO₂ contained in feeds; and, the purified DME has a volume ratio more than 80 vol. %. The separated CO₂ 42 can be recycled back to the gasifier, or to be archived, or to be reused for mending global climate change by reducing carbon emission.

(e) Converting gasoline 5: A zeolite-series catalytic reactor is used as a gasoline conversion reactor, where the purified DME 43 is processed through a dehydration reaction under a reaction temperature set at 250° C.˜350° C. and a reaction pressure controlled at 1 atm˜10 atm to produce H₂O, gasoline-range hydrocarbon compounds and light hydrocarbon compounds.

(f) Further separating out product 6: A flash tower, decanter or other condensing device is used for separating water, the gasoline-range hydrocarbon compounds (around C5˜C10) and light hydrocarbon compounds (around C1˜C4) through condensation. Thus, liquid-phase materials of H₂O and gasoline and gas-phase materials of light hydrocarbon compounds 63 (e.g. liquefied petroleum gas, LPG) are obtained. A 0 vol. %˜99 vol. % of the light hydrocarbon compounds 63 is flown back to process step (e), where the amount of the light hydrocarbon compounds 63 being flown back is controlled at 0 vol. %˜99 vol.% of the total gas-phase flow ratio. Thus, a selectivity rate of a gasoline-range product is increased for obtaining a high-octane gasoline oil. The separation of CO₂ in advance can reduce the volume of gas required for the conversion in the reactor. Furthermore, because the CO₂ concentration of the rear-end LPG product is not high, the LPG obtained has high purity without further purification; can be sold directly; increases the overall value of the product; and, the gasoline fabricated is a high-quality green fuel with high yield, high octane rate and low air pollution of nitrogen and sulfur.

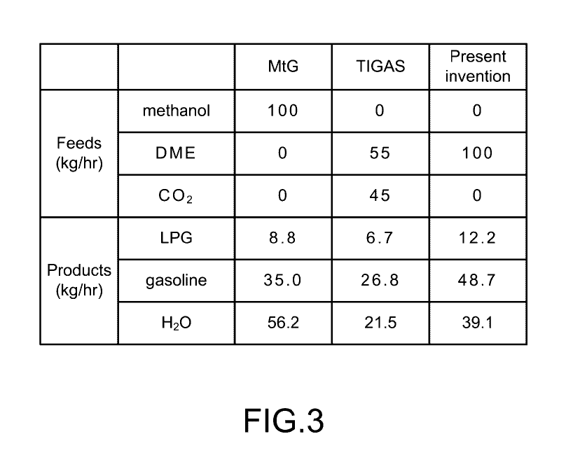

A view for product distributions of MtG, TIGAS and the present invention under the same feed flow rate is shown in FIG. 3. Under the same feed flow rate, the yield of gasoline of the present invention is 1.4 times to that of the MtG. Under in the same volume of the gasoline reactor, the gasoline production of the present invention is 1.8 times to that of the TIGAS.

To sum up, the present invention is a method of fabricating an oil product of gasoline, where the feed rate is significantly reduced; the required volume of a reactor is reduced; the investment cost of hardware is reduced; the separated CO₂ can be recycled back to the gasifier for reuse, storage or other use; CO₂ together with methane and water can be decomposed into a syngas by using a novel high-temperature plasma torch with a regulated hydrogen-carbon ratio of the syngas of biomass for helping the subsequent chemical synthesis reactions; and the final gasoline product has high yield and high octane rate and is a high-quality green fuel with low pollution of sulfur and nitrogen.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of fabricating an oil product of gasoline, comprising steps of:
(c) using a synthesis gas (syngas) of hydrogen (H₂) and carbon monoxide (CO) to obtain dimethyl ether (DME) through a one-step synthesis with a synthesis catalyst;
(d) separating carbon dioxide (CO₂) from DME to obtain purified DME while flowing back unreacted part of said syngas of H₂/CO;
(e) processing dehydration to said purified DME in a reactor to obtain water (H₂O), gasoline-range hydrocarbon compounds and light hydrocarbon compounds; and
(f) separating H₂O, said gasoline-range hydrocarbon compounds and said light hydrocarbon compounds through condensation to obtain liquid-phase materials of H₂O and gasoline and gas-phase materials of light hydrocarbon compounds,
wherein a part of said gas-phase light hydrocarbon compounds is flown back to process step (e) to increase selectivity rate of said gasoline-range hydrocarbon compounds.

2. The method according to claim 1,
wherein, in step (c), said syngas of H₂/CO has a molar ratio of 0.7˜2.5 and is directed to said reactor to process said one-step synthesis under a reaction temperature of 200° C.˜300° C. and a reaction pressure of 30 atm˜60 atm.

3. The method according to claim 1,
wherein, in step (d), a first distillation column and a second distillation column are used to separate CO₂; said unreacted syngas is flown back through said first distillation column to process step (c); and CO₂ is separated in said second distillation column; and
wherein said separated CO₂ has a molar ratio of flow not lower than 90% of a molar ratio of flow of CO₂ contained in feeds; and said purified DME has a volume ratio more than 80 vol. %.

4. The method according to claim 1,
wherein, in step (e), said reactor is a zeolite-series-catalytic reactor used as a gasoline conversion reactor for reaction at a reaction temperature of 250° C.˜350° C. and a controlled reaction pressure of 1 atm˜10 atm.

5. The method according to claim 1,
wherein, in step (f), a condensing and separating device selected from a group consisting of a flash tower and a decanter is used to separate H₂O, said gasoline-range hydrocarbon compounds and said light hydrocarbon compounds; 0 wt %˜89 wt % of said light hydrocarbon compounds is flown back to process step (e); and the amount of said light hydrocarbon compounds being flown back is controlled at 0 vol. %˜99 vol.% of a total gas-phase flow ratio.

6. The method according to claim 1,
wherein, before step (c), step (a) and step (b) are further processed; and
wherein step (a) is a process of plasma-assisted gasification and step (b) is a process of gas purification and adjustment.

7. The method according to claim 6,
wherein, in step (a), a raw syngas comprising a biomass and an oxidizer is obtained through said plasma-assisted gasification; a temperature of a furnace used in said plasma-assisted gasification is controlled by a power of a plasma torch and a supply amount of said oxidizer; and said plasma-assisted gasification is processed at a temperature of 850° C.˜1400° C.

8. The method according to claim 7,
wherein said oxidizer is selected from a group consisting of CO₂, H₂O, oxygen (O₂), air and a mixture of elements selected from CO₂, H₂O, O₂ and air; and
wherein said raw syngas is a mixture of CO, H₂, CO₂ and H₂O.

9. The method according to claim 6,
wherein, in step (b), acidic gases and compounds containing nitrogen, sulfur and/or chlorine are removed from said raw syngas to obtain said syngas which contains CO₂ less than 10 vol. %, nitrogen less than 1 ppm, sulfur less than 1 ppm and chlorine less than 1 ppm; and said syngas has a molar ratio of H₂/CO at 0.7˜2.5.
